(19) United States
(12) Patent Application Publication
(10) Pub. No.: US 2025/0186030 A1
(43) Pub. Date: Jun. 12, 2025

(54) BIOPSY CAP ASSEMBLIES

(71) Applicant: Boston Scientific Medical Device Limited, Galway (IE)

(72) Inventors: Amit BHAROS, Jabalpur (IN); Priyanshu GUPTA, Ujjain (IN); Mahendra Pratap SINGH, Gurgaon (IN); Neeraj GARG, Gurgaon (IN)

(73) Assignee: Boston Scientific Medical Device Limited, Galway (IE)

(21) Appl. No.: 18/973,934

(22) Filed: Dec. 9, 2024

Related U.S. Application Data
(60) Provisional application No. 63/608,886, filed on Dec. 12, 2023.

(51) Int. Cl. A61B 10/04; A61B 1/00

(52) U.S. Cl. CPC A61B 10/04; A61B 1/00042; A61B 1/00137

(57) ABSTRACT

A biopsy cap assembly may include a housing and a sleeve positioned within the housing. The sleeve may include a first plurality of threads. The biopsy cap assembly may also include a knob positioned around the sleeve. The knob may include a second plurality of threads that engage with the first plurality of threads. Rotation of the knob relative to the housing and the sleeve may cause a portion of the sleeve to decrease in diameter.

### CROSS-REFERENCE TO RELATED APPLICATIONS

This application claims the benefit of priority under 35 U.S.C. § 119 from U.S. Provisional Application No. 63/608,886, filed Dec. 12, 2023, which is incorporated by reference herein in its entirety.

### TECHNICAL FIELD

Various aspects of this disclosure relate generally to devices and methods for accessories for medical devices, and, in particular, to biopsy cap assemblies that are couplable to variety of biopsy ports of medical devices.

### BACKGROUND

Medical devices, such as scopes (e.g., endoscopes, duodenoscopes, etc.), may include a handle and a sheath/shaft, with the shaft being insertable into a body lumen of a subject. The shaft may terminate in a distal tip portion, which may include features such as elevators, optical elements (e.g., camera, lighting, etc.), air/water outlets, working channel openings, and/or accessory devices. Actuators in the handle of the scope may control actuatable elements of the shaft and/or distal tip. For example, buttons, knobs, levers, etc. may control elements of the shaft and/or distal tip. A working channel may extend from the handle, through the shaft, to the distal tip. A proximal end of the working channel may be coupled to and in fluid communication with a biopsy port of the handle. During a medical procedure, a variety of accessory devices may be passed through the biopsy port and through the shaft, to the distal tip.

### SUMMARY

Each of the aspects disclosed herein may include one or more of the features described in connection with any of the other disclosed aspects.

Aspects of the disclosure relate to, among other things, systems, devices, and methods for biopsy cap assemblies.

A biopsy cap assembly may include a housing and a sleeve positioned within the housing. The sleeve may include a first plurality of threads. The biopsy cap assembly may also include a knob positioned around the sleeve. The knob may include a second plurality of threads that engage with the first plurality of threads. Rotation of the knob relative to the housing and the sleeve may cause a portion of the sleeve to decrease in diameter.

Any of the assemblies described herein may have any of the following features, alone or in any combination. The rotation of the knob relative to the housing and the sleeve may cause the sleeve to move proximally. A distal portion of the sleeve may have a greater outer diameter than a proximal portion of the sleeve. An outer surface of the sleeve may taper radially outward in a distal direction. The knob may have a tubular wall. The tubular wall may extend circumferentially around the sleeve. The sleeve may include a distal portion having a plurality of fins around a central opening. The rotation of the knob relative to the housing and the sleeve may cause the plurality of fins to move closer to one another, thereby decreasing a diameter of the central opening. The plurality of fins may include at least four fins. Slits may extend radially outward from the central opening, between adjacent fins of the plurality of fins. The housing may include a slot. The knob may include a protrusion that extends through the slot. The protrusion may be configured to be contacted and moved by a user. The assembly may further comprise a biopsy cap body positioned within the housing. The assembly may further comprise a gasket positioned at least partially within the biopsy cap body. The gasket may include a distal flange that contacts a distal end of the sleeve. The sleeve may include a plurality of longitudinally extending recesses. The sleeve may include a wall. A proximal portion of the wall may be thinner than a distal portion of the wall.

In another example, a biopsy cap assembly may include a housing and a sleeve positioned within the housing. The sleeve may have an outer surface that tapers radially outward in a distal direction. The assembly may further comprise a knob positioned around the sleeve. Rotation of the knob relative to the housing and the sleeve may cause the sleeve to move proximally relative to the knob, thereby increasing an interference between the knob and the sleeve.

Any of the assemblies described herein may have any of the following features, alone or in any of combination. The sleeve may include a distal portion having a plurality of fins around a central opening. The rotation of the knob relative to the housing and the sleeve may cause the plurality of fins to move closer to one another.

An exemplary method of coupling a biopsy cap assembly to a biopsy port of a medical device may include: positioning a distal portion of the biopsy cap assembly over the biopsy port; and rotating a knob of the biopsy cap assembly relative to a housing of the biopsy cap assembly in order to inhibit removal of the biopsy cap assembly from the biopsy port.

Any of the methods disclosed herein may have any of the following features, alone or in any combination. Rotating the knob may cause a sleeve of the biopsy cap assembly to tighten around the biopsy port. Rotating the knob of the biopsy cap assembly may include contacting a protrusion of the knob that extends through a slot of the housing of the biopsy cap assembly and moving the protrusion along the slot.

The aspects discussed above may be combined in any suitable combination or sub-combination.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 depicts an exemplary medical device in accordance with aspects of the disclosure.

FIG. 2 depicts a side view of a biopsy cap assembly for use with the medical device of FIG. 1 and other medical devices.

FIG. 3 is an exploded view of the biopsy cap assembly of FIG. 2.

FIG. 4 is a cross-sectional view of the biopsy cap assembly of FIGS. 2 and 3.

FIG. 5A shows a cross-sectional view of the biopsy cap assembly of FIGS. 2-4 in a first configuration on a biopsy port of a medical device, such as the medical device of FIG. 1.

FIG. 5B shows a cross-sectional view of the biopsy cap assembly of FIGS. 2-4 in a second configuration.

FIG. 6 depicts a bottom view of a sleeve of the biopsy cap assembly of FIGS. 2-4.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator/toward a treatment site, and the term “proximal” refers to a direction toward an operator. One or more of the drawings include arrows labeled “P” and “D,” referring to proximal and distal directions, respectively. The term “exemplary” is used in the sense of “example,” rather than “ideal.” The term “approximately,” or like terms (e.g., “substantially”), includes values +/−10% of a stated value.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

A biopsy cap assembly may be coupled to a biopsy port of a medical device (for example, a scope of any type). The biopsy cap assembly may be universal. In other words, the biopsy cap assembly may be removably couplable to any biopsy port of any medical device. Alternatively, the biopsy cap assembly may not be universal (may not work with a subset of biopsy ports) but may be couplable to a wide variety of biopsy ports of a variety of medical devices. The biopsy cap assembly may include multiple components housed in a single housing.

The biopsy cap assembly may include a biopsy cap and a housing. The biopsy cap may be flexible and may permit passage of an accessory device through the biopsy cap and into a working channel of a medical device. The biopsy cap assembly may also include a sleeve that may function as a neck gripper to grip a neck of a biopsy port. The biopsy cap assembly may also include a knob that may be rotated in order to tighten the sleeve on the biopsy port and help affix the biopsy cap assembly to the biopsy port. For example, the sleeve and the knob may include threads. In some examples, the sleeve may include a plurality of fins that may grip onto the housing as the sleeve is compressed radially inward.

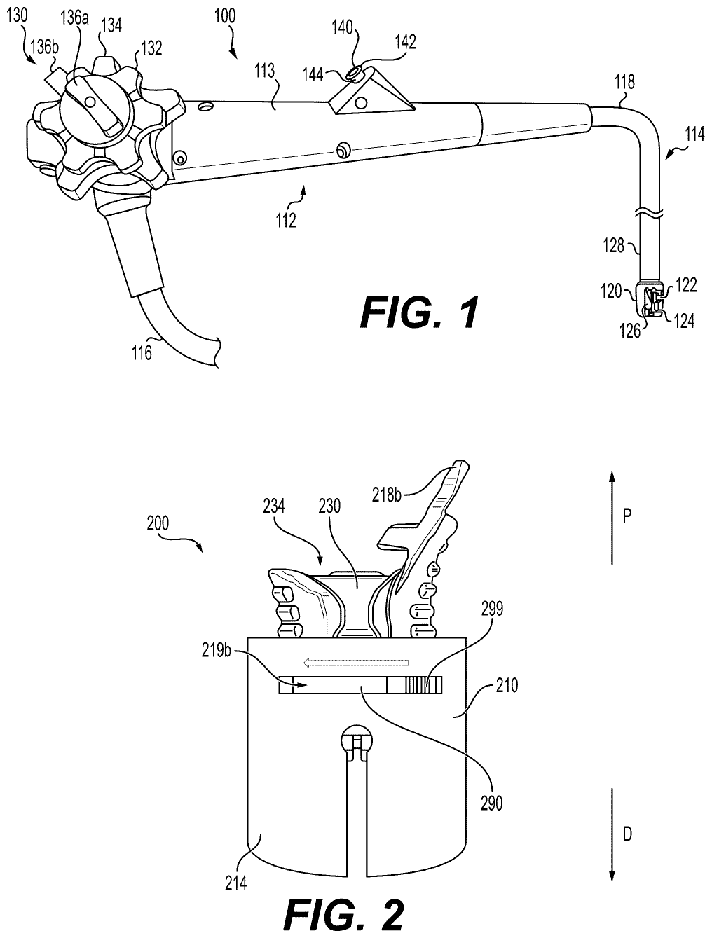

FIG. 1 depicts an exemplary medical device 100 (e.g., a duodenoscope) having a handle 112 and an insertion portion 114. Medical device 100 may also include an umbilicus 116 for purposes of connecting medical device 100 to sources of, for example, air, water, suction, power, etc., as well as to image processing and/or viewing equipment.

Insertion portion 114 may include a sheath or shaft 118 and a distal tip 120. Distal tip 120 may include an imaging device 122 (e.g., a camera) and a lighting source 124 (e.g., an LED or an optical fiber). Distal tip 120 may be side-facing. That is, imaging device 122 and lighting source 124 may face radially outward, perpendicularly, approximately perpendicularly, or otherwise transverse to a longitudinal axis of shaft 118 and distal tip 120. Alternatively, distal tip 120 may be forward-facing (imaging device 122 and/or lighting source 124 may face distally).

Distal tip 120 may also include an actuatable element, for example an elevator 126 for changing an orientation of a tool inserted in a working channel of medical device 100. Elevator 126 may alternatively be referred to as a swing stand, pivot stand, raising base, or any suitable other term. Elevator 126 may be pivotable via, for example, an actuation wire or another control element that extends from handle 112, through shaft 118, to elevator 126.

A distal portion of shaft 118 that is connected to distal tip 120 may have a steerable section 128. Steerable section 128 may include, for example, an articulation joint, such as the articulation joints discussed below. Shaft 118 and steerable section 128 may include a variety of structures which are known or may become known in the art. In some examples, one or more steering wires may be coupled to steerable section 128 so that, as the steering wires are tensioned/de-tensioned, steerable section 128 articulates in one or more directions.

Handle 112 may have a housing 113 that encases/houses various elements of handle 112. Handle 112 may have one or more actuators/control mechanisms 130. Control mechanisms 130 may provide control over steerable section 128 or may allow for provision of air, water, suction, etc. For example, handle 112 may include a first control knob 132 and a second control knob 134 for left, right, up, and/or down control of steerable section 128. For example, first knob 132 may provide left/right control of steerable section 128, and second knob 134 may provide up/down control of steerable section 128. Handle 112 may further include one or more locking mechanisms 136a, 136b (e.g., knobs or levers) for preventing steering of steerable section 128 in at least one of an up, down, left, or right direction. Handle 112 may include an elevator control mechanism (not shown). A port 140 may allow passage of a tool through port 140, into a working channel of the medical device 100, through shaft 118, to distal tip 120. Port 140 may have a flange 142 and a neck 144, as described in further detail below.

In use, an operator may insert at least a portion of shaft 118 into a body lumen of a subject. Distal tip 120 may be navigated to a procedure site in the body lumen. The operator may use knobs 132, 134 to steer steerable section 128 of shaft 118 to a desired position. The operator may insert an accessory device, such as an instrument (not shown) into port 140, and pass the tool through shaft 118 via a working channel to distal tip 120. The tool may exit the working channel at distal tip 120. The operator may use the elevator control mechanism (not shown) to raise elevator 126 and angle the accessory device toward a desired location (e.g., a papilla of the pancreatico-biliary tract). The operator may use the accessory device to perform a medical procedure.

Although FIG. 1 depicts a side-viewing duodenoscope, it will be appreciated that, as discussed above, this disclosure is not so limited. The aspects discussed herein may alternatively be utilized with other types of medical devices. For example, the embodiments disclosed herein may be utilized with any type of scope.

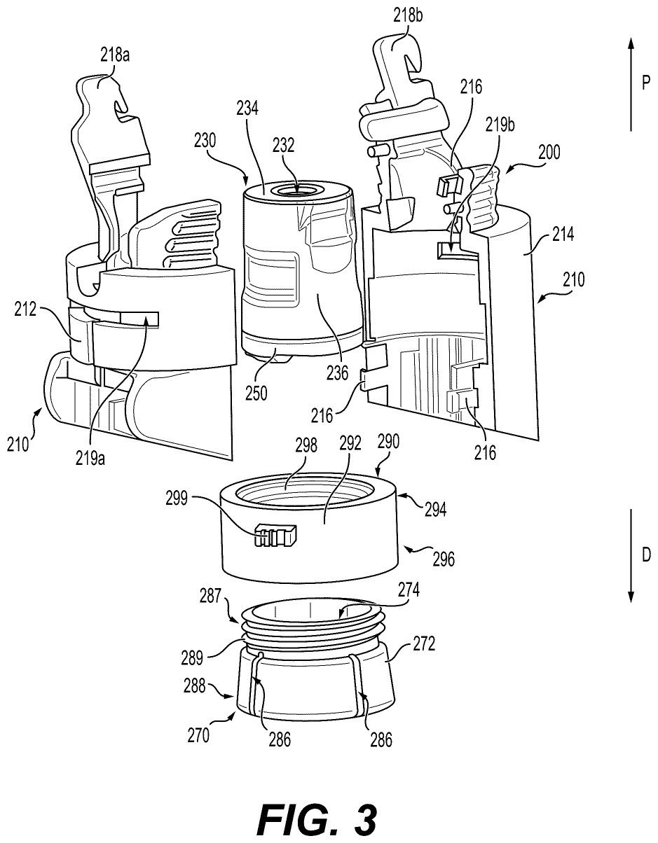

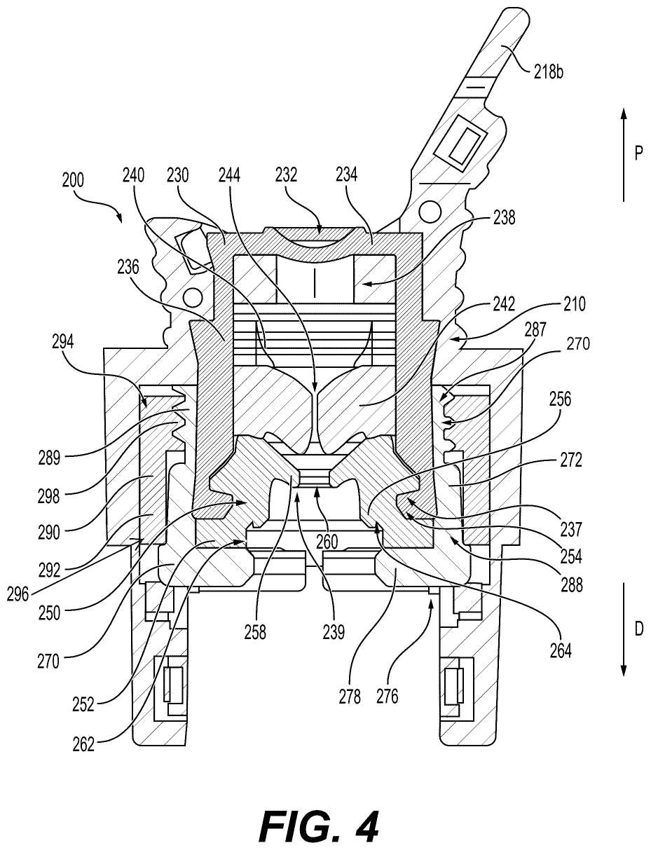

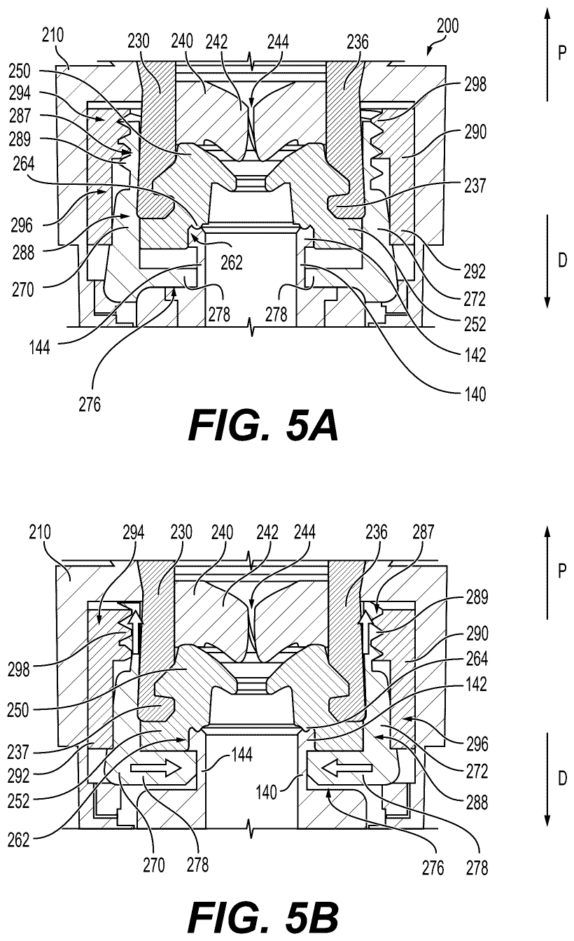

FIG. 2 shows a biopsy cap assembly 200. FIG. 3 shows an exploded view of biopsy cap assembly 200. FIG. 4 shows a cross-sectional view of biopsy cap assembly 200. FIGS. 5A and 5B show biopsy cap assembly 200 positioned on port 140 of medical device 100, with biopsy cap assembly 200 in a first configuration and a second configuration, respectively. Port 140 is merely exemplary, and biopsy cap assembly 200 may be used with a variety of ports of medical devices (e.g., scopes).

Biopsy cap assembly 200 may include a housing 210, a biopsy cap body 230, an insert 240, a gasket 250, a sleeve 270, and a knob 290. The various elements of biopsy cap assembly 200 may be contained by housing 210 and may be pre-packaged within housing 210. in these aspects, a user may not assemble biopsy cap assembly 200 and may use the entire assembly 200 after it has already been assembled within housing 210.

Housing 210 may include a first piece 212 (e.g., a first half) and a second piece 214 (e.g., a second half). Second piece 212 may include a plurality of protrusions 216 that may mate with a plurality of recesses (not shown) of first piece to couple (e.g., fixedly couple) first piece 212 to second piece 214. First piece 212 may include a first hook 218a, and second piece 214 may include a second hook 218b. Hooks 218a and 218b may be configured to receive one or more accessory devices (e.g., guidewires) to help retain the accessory device during a medical procedure. Hooks 218a, 218b may facilitate rapid exchange of accessories during the medical procedure.

Biopsy cap body 230 may be disposed within assembled housing 210. Biopsy cap body 230 may include any of the features of any biopsy cap known in the art. Biopsy cap body 230 may include an opening 232 in a proximal wall 234 of biopsy cap body 230. Proximal wall 234 may be substantially flat. In some examples, opening 232 may be a slit that is configured to deform around an accessory device inserted into opening 232 and to inhibit leakage of fluid from the working channel out of opening 232. Biopsy cap body 230 may include an outer wall 236, which may have a substantially tubular shape. Outer wall 236 and proximal wall 234 may define an inner chamber 238 (FIG. 4). Chamber 238 may have an open distal end 239.

As shown in FIGS. 4, 5A, and 5B, an insert 240 may be disposed within biopsy cap body 230 (within chamber 238). Insert 240 may include one or more lips 242. In some example, a plurality of lips 242 may be positioned around an inner perimeter of insert 240. In other examples, a single lip 242 may extend around an inner perimeter (e.g., an inner circumference) of insert 240. An opening 244 may extend between lip(s) 242. An accessory device (e.g., a shaft of an accessory device) may pass through the opening, and lip(s) 242 may help to form a seal around the accessory device or function as a valve, inhibiting flow of fluid in a proximal direction, out of a proximal opening of biopsy cap body 230.

Gasket 250 may be disposed in/on a distal portion of biopsy cap body 230. In some aspects, gasket 250 is at least partially within biopsy cap body 230. For example, gasket 250 may extend through open distal end 239. Gasket 250 may include a distal flange 252, which may rest against and contact a distal end of outer wall 236 of biopsy cap body 230.

Gasket 250 may also include a recess 254 formed in an outer surface of an outer wall 256 of gasket 250. In some examples, recess 254 may be an annular recess. Alternatively, outer wall 256 may include a plurality of recesses 254 formed in the outer surface of outer wall 256. Outer wall 236 of biopsy cap body 230 may include a protrusion 237 extending radially inward from an inner surface of outer wall 236. In some examples, protrusion 237 may have an annular shape and may extend around an inner circumference of inner wall 236. Alternatively, wall 236 may include a plurality of separate protrusions 237 extending radially inward from an inner surface of inner wall 236. Protrusion 237 may be received within recess 254 of gasket 250, thereby securing gasket 250 to biopsy cap body 230.

Gasket 250 may include one or more lips 258 (labeled in FIG. 4), which may have any of the properties of lip(s) 242 of insert 240. Lip(s) 258 may extend radially inward from outer wall 256 of gasket 250. Similarly to lip(s) 242, lip(s) 258 may have a central opening 260 (labeled in FIG. 4) extending through lip(s) 258. An accessory device inserted into opening 232 of biopsy cap body 230 may extend through opening 244 of insert 240 and opening 260 of gasket 250. Lip(s) 258 may help to form a seal against the accessory device (e.g., against a shaft of the accessory device), inhibiting proximal leakage of fluid.

A radially inner surface of wall 256 of gasket 250 may include one or more features configured to mate with a biopsy port, such as biopsy port 140 (see FIGS. 1, 5A, and 5B). The configuration of biopsy port 140 is merely exemplary, and biopsy cap assembly 200, including gasket 250, may be configured to be removably attached to various different configurations of biopsy ports. As shown in FIGS. 1, 5A and 5B, in particular, port 140 may include a flange 142 and a neck 144. Wall 256 of gasket 250 may have a recess 262 formed therein. In some examples, recess 262 may have an annular shape. Recess 262 may be formed on a distalmost end of wall 256, such that recess 262 has an open distal side. A proximal wall of recess 262 may have a protrusion 264 extending distally from the proximal wall. As shown in FIGS. 5A and 5B, flange 142 may be received within recess 262, with a proximal surface of flange 142 abutting a proximal surface of recess 262 and/or protrusion 264. Thus, gasket 250 may help to seat biopsy cap assembly 200 on port 140.

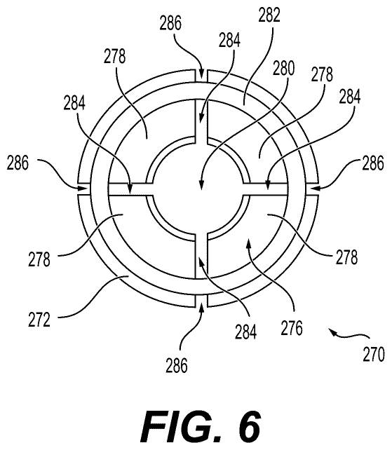

Sleeve 270 may extend about a proximal portion of biopsy cap body 230 (and gasket 250). FIG. 6 shows a plan view of sleeve 270 to show details of sleeve 270. Sleeve 270 may include an outer wall 272, which may have an approximately tubular shape. Outer wall 272 may define a cavity 274 (FIG. 3). Cavity 274 may have an open proximal end.

As shown particularly in FIG. 6, a distal end 276 of sleeve 270 may include a plurality of fins 278. Although four fins 278 are shown in FIG. 6, any suitable number of fins may be used. Each fin 278 may extend from outer wall 272 to a central opening 280. Although fins 278 are shown as being disposed at a distalmost end of sleeve 270, fins 278 may extend from a more proximal portion of sleeve 270. Fins 278 may be joined together at their radially outermost ends by an outer band 282 of distal end 276. Slits 284 may extend radially outward from opening 280 toward outer band 282 but may not extend through outer band 282. In alternatives, outer band 282 may be omitted. Each fin 278 may have a shape like a segment of an annulus. Fins 278 may all have the same shape or may have different shapes. Distal end 276 may be made from a different material (e.g., a more flexible material) than other portions of sleeve 270 (e.g., outer wall 272). In some examples, distal end 276 may be formed from a separate material than outer wall 272, and outer band 282 may be coupled to/affixed to outer wall 272 (e.g., via overmolding, adhesive, or other mechanisms). In other examples, outer wall 272 and distal end 276 (including fins 278) may be formed from a single, integral piece of material.

As shown in FIGS. 3-5B, a proximal portion 287 of wall 272 may have a decreased radial thickness (a thickness between outer and inner radiuses of wall 272) as compared to a distal portion 288 of wall 272. In other words, distal portion 288 may have a greater wall thickness (may be thicker) than proximal portion 287. Wall 272 at proximal portion 287 may be thinner than wall 272 at distal portion 288. Proximal portion 287 of wall 272 may have one or more threads 289 that extend around proximal portion 287.

As shown in FIG. 3, distal portion 288 may have a plurality of recesses 286 formed in an outer surface of outer wall 272. Recesses 286 may extend longitudinally or approximately longitudinally, approximately parallel to a central longitudinal axis of biopsy cap assembly 200 (in a proximal/distal direction). In some examples, recesses 286 may be circumferentially/angularly aligned with slits 284. Recesses 286 may impart flexibility to outer wall 272 and particularly to distal portion 288. Distal portion 288 of wall 272 may taper radially outward in a distal direction, such that sleeve 270 may have an increasingly large radius/diameter moving in a distal direction. A distalmost end of sleeve 270 may have a greater outer or inner diameter than a proximal portion of sleeve 270.

Biopsy cap body 230 and gasket 250 may be received within (positioned within) cavity 274 (labeled in FIG. 3). A distal surface of gasket 250 may rest against distal end 276 of sleeve 270. Flange 252 of gasket 250 may be within distal portion 288 of wall 272. Protrusion 237 of outer wall 236 of biopsy cap body 230 may also be within distal portion 288 of wall 272. extending radially inward from an inner surface of outer wall 236. Biopsy cap body 230 may extend proximally from a proximal open end of cavity 274. In other words, biopsy cap body 230 may extend proximally of a proximalmost end of outer wall 272 of sleeve 270.

Knob 290 may extend around sleeve 270, such that knob 290 is radially outward of biopsy cap body 230 and sleeve 270. Knob 290 may have an approximately tubular outer wall 292. Wall 292 may extend circumferentially around sleeve 270. Wall 292 may have a proximal portion 294 and a distal portion 296. Proximal portion 294 may include a plurality of threads 298 on an inner surface of wall 292. Threads 298 may mate with and interact with threads 289 of sleeve 270. Sleeve 270 may be disposed radially within knob 290, such that distal portion 296 of knob 290 is adjacent to distal portion 288 of sleeve 270 and such that threads 298 of knob 290 mate with threads 289 of sleeve 270. In some examples, knob 290 may be formed from a rigid material. In other examples, knob 290 may be flexible or may include a combination of rigid and flexible materials.

As shown in FIGS. 2 and 3, knob 290 may include a protrusion 299. Protrusion 299 may extend radially outward from a radially outer surface of wall 292. Protrusion 299 may have grooves or other grip-enhancing structures formed on an outer surface of protrusion 299. Protrusion 299 may be configured to be contacted by a finger of a user in order to change a configuration of biopsy cap assembly 200, as described below. Although one protrusion 299 is depicted, it will be appreciated that knob 290 may include a plurality of protrusions. In some aspects, knob 290 may include two protrusions that are circumferentially spaced apart, for example, diametrically opposed to one another (approximately 180 degrees apart from one another).

As shown in FIG. 2, housing 210 may include one or more slots. For example, the slots may extend around a portion of a circumference of housing 210. For example, first piece 212 of housing 210 may include a first slot 219a, and second piece 214 of housing 210 may include a second slot 219b. Each of lots 219a, 219b may extend around approximately one quarter to one third of a circumference of housing 210 (e.g., around one half to two thirds of each piece 212, 214). An entirety of slots 219a, 219b may be on one of first piece 212 or second piece 214, respectively. Although one protrusion 299 is shown in the Figures, it will be appreciated that knob 290 may include more than one protrusions 299 (e.g., two protrusions 299). Each protrusion 299 may extend through a respective slot 219a, 219b, such that each protrusion 299 may be contacted by a finger of a user. For example, protrusion 299 may extend through slot 219b, as shown in FIG. 2. By exerting a circumferential, rotational force on protrusion 299 (as shown by the arrow in FIG. 2), the user may move rotate knob 290 by moving protrusion 299 along slot 219b (or slot 219a).

FIGS. 5A and 5B show biopsy cap assembly 200 positioned on port 140 in a first, relaxed configuration (FIG. 5A) and a second, tightened configuration (FIG. 5B). In use, an operator may position biopsy cap assembly 200 over port 140 by inserting flange 142 into opening 280 at a distal end of sleeve 270 with biopsy cap assembly 200 in the first configuration of FIG. 5A. When positioning biopsy cap assembly 200 on port 140, protrusion 299 may be at a first end of slot 219b, as shown in FIG. 2. Biopsy cap assembly 200 may be moved distally over port 140 until flange 142 is abutting gasket 250, as described above and shown in FIG. 5A. Neck 144 may extend through opening 280 of sleeve 270 and into a distal portion of cavity 274 (shown in FIG. 3) of sleeve 270. Fins 278 may be disposed at a distal portion of neck 144 (or at another portion of neck 144) and may be adjacent to a surface of handle 112 that is coupled to the distal portion of neck 144.

As shown in FIG. 5A, when biopsy cap assembly 200 is in a relaxed configuration (e.g., when protrusion 299 is at the first end of slot 219b, as shown in FIG. 2), knob 290 and sleeve 270 may have no interference with one another or little interference with one another, such that fins 278 are spaced apart from one another and opening 280 has an open, relaxed configuration. In the first configuration, biopsy cap assembly 200 may be removable from port 140.

A user may contact protrusion 299 and move it along slot 219b to rotate knob 290 relative to housing 210. As knob 290 moves, it may be inhibited from moving proximally or distally because protrusion 299 remains within slot 219b. Threads 298 of knob 290 may engage with threads 289 of 270, which may cause sleeve 270 to move proximally (upward in FIGS. 5A and 5B), as shown by the arrows in FIG. 5B. Proximal/upward movement of sleeve 270 relative to knob 290 may cause an increasingly distal portion of outer wall 272 of sleeve 270 to engage wall 292 of knob 290. Because outer wall 272 tapers radially outward in a distal direction, interference may increase between wall 292 of knob 290 and outer wall 272 of sleeve 270 as sleeve 270 moves proximally/upward. In some examples, one or both of outer wall 272 and wall 292 may comprise flexible material that may have frictional interference with one another. Alternatively, knob 290 may be formed of a rigid material. Outer wall 272 of sleeve 270 may be formed from a rigid material, but outer wall 272 may have flexibility imparted to it by recesses 286.

FIG. 5B shows a tightened configuration of biopsy cap assembly 200, in which protrusion 299 may be on a second end of slot 219b (an opposite end of slot 219b that is shown in FIG. 2). As shown in FIG. 5B, wall 292 of knob 290 may exert a radially inward force on outer wall 272 (e.g., distal portion 288 of outer wall 272) and distal end 276, including fins 278. Recesses 286 and slits 284 may facilitate radially inward movement of distal portions of sleeve 270, including for example, distal portion 288. In the tightened configuration of FIG. 5B, portions of sleeve 270 (e.g., distal portion 288 of outer wall 272, distal end 276, and/or opening 280) may have a decreased diameter relative to the relaxed configuration of FIG. 5A.

Fins 278 may move radially inward to tighten around neck 144, and opening 280 may decrease in radius/diameter. Radially inner edges of fins 278 may move closer to one another. The arrows in FIG. 5B show the inward movement of fins 278 and/or outer wall 272 (e.g., distal portion 288). Fins 278 may be sufficiently flexible and sufficiently resilient that they exert a retaining force on neck 144. In the tightened configuration of FIG. 5B, biopsy cap assembly 200 may be inhibited from unintentional removal from port 140. Sleeve 270 may be akin to a collet, tightening around port 140 via fins 278.

Biopsy cap assembly 200 may be usable with a variety of biopsy ports 140, having a variety of outer diameters of flange 142 and/or outer diameters of neck 144, as well as varying heights of neck 144 (varying distances between a body of handle 112 and a proximal surface of flange 142). For example, opening 280 may have a sufficient size in the relaxed configuration of FIG. 5A to fit over a wide variety of ports 140 (for example, opening 280 may be wider than flange 142 in the relaxed configuration of FIG. 5A). A flexibility and size of fins 278, outer wall 272, and knob 290 may be such that fins 278 may engage and retain a wide variety of sizes of necks 144.

To remove biopsy cap assembly 200 from port 140, protrusion 299 may be rotated in a direction that is opposite of the direction used to tighten biopsy cap assembly 200 (transition biopsy cap assembly 200 from the configuration of FIG. 5A to the configuration of FIG. 5B). This rotation may have an opposite effect of the tightening described above. As protrusion 299 is rotated, sleeve 270 may move distally/downward, thereby decreasing the radially inward force from knob 290 and moving fins 278 outward relative to one another. A diameter of opening 280 may increase. Biopsy cap assembly 200 may be pulled from (e.g., proximally) or otherwise removed port 140.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

What is claimed is:

1. A biopsy cap assembly including:
a housing;
a sleeve positioned within the housing, wherein the sleeve includes a first plurality of threads; and
a knob positioned around the sleeve, wherein the knob includes a second plurality of threads that engage with the first plurality of threads;
wherein rotation of the knob relative to the housing and the sleeve causes a portion of the sleeve to decrease in diameter.

2. The biopsy cap assembly of claim 1, wherein the rotation of the knob relative to the housing and the sleeve causes the sleeve to move proximally.

3. The biopsy cap assembly of claim 2, wherein a distal portion of the sleeve has a greater outer diameter than a proximal portion of the sleeve.

4. The biopsy cap assembly of claim 3, wherein an outer surface of the sleeve tapers radially outward in a distal direction.

5. The biopsy cap assembly of claim 4, wherein the knob has a tubular wall, wherein the tubular wall extends circumferentially around the sleeve.

6. The biopsy cap assembly of claim 1, wherein the sleeve includes a distal portion having a plurality of fins around a central opening.

7. The biopsy cap assembly of claim 6, wherein the rotation of the knob relative to the housing and the sleeve causes the plurality of fins to move closer to one another, thereby decreasing a diameter of the central opening.

8. The biopsy cap assembly of claim 6, wherein the plurality of fins includes at least four fins.

9. The biopsy cap assembly of claim 6, wherein slits extend radially outward from the central opening, between adjacent fins of the plurality of fins.

10. The biopsy cap assembly of claim 1, wherein the housing includes a slot, wherein the knob includes a protrusion that extends through the slot, and wherein the protrusion is configured to be contacted and moved by a user.

11. The biopsy cap assembly of claim 1, further comprising a biopsy cap body positioned within the housing.

12. The biopsy cap assembly of claim 11, further comprising a gasket positioned at least partially within the biopsy cap body.

13. The biopsy cap assembly of claim 12, wherein the gasket includes a distal flange that contacts a distal end of the sleeve.

14. The biopsy cap assembly of claim 1, wherein the sleeve includes a plurality of longitudinally extending recesses.

15. The biopsy cap assembly of claim 1, wherein the sleeve includes a wall, and wherein a proximal portion of the wall is thinner than a distal portion of the wall.

16. A biopsy cap assembly including:
a housing;
a sleeve positioned within the housing, wherein the sleeve has an outer surface that tapers radially outward in a distal direction; and
a knob positioned around the sleeve;
wherein rotation of the knob relative to the housing and the sleeve causes the sleeve to move proximally relative to the knob, thereby increasing an interference between the knob and the sleeve.

17. The biopsy cap assembly of claim 16, wherein the sleeve includes a distal portion having a plurality of fins around a central opening, and wherein the rotation of the knob relative to the housing and the sleeve causes the plurality of fins to move closer to one another.

18. A method of coupling a biopsy cap assembly to a biopsy port of a medical device, the method including:
positioning a distal portion of the biopsy cap assembly over the biopsy port; and
rotating a knob of the biopsy cap assembly relative to a housing of the biopsy cap assembly in order to inhibit removal of the biopsy cap assembly from the biopsy port.

19. The method of claim 18, wherein rotating the knob causes a sleeve of the biopsy cap assembly to tighten around the biopsy port.

20. The method of claim 18, wherein rotating the knob of the biopsy cap assembly includes contacting a protrusion of the knob that extends through a slot of the housing of the biopsy cap assembly and moving the protrusion along the slot.
